(19) United States
(12) Patent Application Publication
(10) Pub. No.: US 2003/0097223 A1
(43) Pub. Date: May 22, 2003

(54) Primer design system

(75) Inventors: Hiroki Nakae, Fujisawa (JP); Sigeo Ihara, Tokorozawa (JP)

Correspondence Address: REED SMITH HAZEL THOMAS LLP, Suite 1400, 3110 Fairview Park Drive, Falls Church, VA, 22042, US

(73) Assignee: Hitachi, Ltd.

(21) Appl. No.: 10/223,374

(22) Filed: Aug. 20, 2002

Related U.S. Application Data
(63) Continuation-in-part of application No. 09/527,440, filed on Mar. 17, 2000.

(30) Foreign Application Priority Data
Dec. 14, 1999 (JP) 11-355080

(51) Int. Cl. G06G 7/48; G06G 7/58; G06F 19/00; G01N 33/48; G01N 33/50

(52) U.S. Cl. 702/20; 703/11

(57) ABSTRACT

A primer design system in which DNA nucleotide sequences are obtained from a database comprising a plurality of different DNA nucleotide sequences, and the nucleotide sequences of primers capable of hybridizing specifically to the exons predicted from the DNAs thus obtained are determined. A plurality of primers are simultaneously designed by using each of the predicted exons as a template. In addition, specificity evaluation is conducted for the exons, the primers and the primer pairs.

The present application is a continuation-in-part application of the U.S. patent application Ser. No. 09/527,440 filed on Mar. 17, 2000.

### BACKGROUND OF THE INVENTION

1. Field of the Invention

The present invention relates to a technique of DNA analysis, and more particularly to a primer design system, a method for designing primers, a storage medium on which is recorded a program for allowing a computer to function as a primer design system, a storage medium on which is recorded data which are necessary during DNA analysis, plates containing primers which are necessary during DNA analysis, a DNA analysis kit comprising a storage medium and primers which are necessary during DNA analysis, and a method for analyzing DNA.

2. Description of the Related Art

In the 1990's, the human genome project has flourished, leading to an increasingly clearer understanding of the genome sequences for E. coli, yeasts, nematodes, rice, Arabidopsis thaliana, mice, rats, humans, and the like. This has been accompanied by a veritable explosion of highly efficient methods for the analysis of nucleotide sequences as well as the development of techniques such as the computerization of sequence analyses and higher throughput in techniques for the analysis of nucleotide sequences of the gene, YAC and BAC libraries, and chromosome markers.

The recent progress of the genome project and the development of sequence analyzing techniques have resulted in the continuing accumulation of massive gene-related databases (see FIG. 1), making bioinformatics increasingly necessary in the data processing of such massive amounts of gene-related data. Bioinformatics is an expression created from biology and informatics (the science of information), meaning research combining life sciences and information sciences, that is, the comprehensive science of handling biological data in its entirety with the intention of making broader use not only of genome data but of biological data, from genes to protein structure or function. At present, however, bioinformatics is not being adequately used in industry-based genetic functional analysis.

Genomic DNA includes both intron and exon regions. Of these, exons encode proteins, making the analysis of exons extremely important in genetic analysis. However, it is extremely difficult to specify exons that are compatible with the actual purpose of research, and conventional genetic analysis has involved selecting exons compatible with the purpose of research merely through trial and error.

FIG. 7 depicts the course of conventional genetic analysis. Conventionally, the individual genes or proteins of interest are generally identified (step 600) by subtraction or DD based cloning of gene, nucleotide sequences or protein amino acid sequences, and then checked what type of functions they have. That is, exons which are considered compatible with the purpose of research are selected beforehand (step 602) from the identified nucleotide sequences to design corresponding primers (step 603). The primers are then used in PCR (polymerase chain reaction) to amplify the target exons (step 604) for analysis of the exons (step 605). PCR is a method in which primers are designed for both ends of the region that is to be amplified, and genes are amplified logarithmically by temperature cycles using a heat resistant DNA enzyme such as Taq DNA polymerase. Primers are oligonucleotides having an —OH at the 3′ end necessary to initiate DNA synthesis.

When the exons selected by the analysis in step 605 prove to be incompatible with the purpose of the research in such conventional genetic analysis, the process (step 606) must be repeated from the exon selection in step 602, making it extremely important to ensure the reliable selection of exons compatible with the purpose of research. During the analysis of differences in gene levels occurring between normal individuals and patients afflicted with a certain disease (such as cancer), for example, exons which are the target of research will be the exons leading to the disease, but it is extremely difficult to determine which exons are the exons in question, and there has been no other way to analyze candidate exons other than by the trial and error described above in order to determine such exons.

### SUMMARY OF THE INVENTION

The present invention is intended to provide a method for more efficiently designing primers for various genes of interest, which has been an inefficient undertaking in the past because of the extreme difficulty involved in specifying desired exons as described above.

More specifically, an object of the present invention is to provide a high-throughput method for genetic functional analysis which is completely different from conventional methods, by making use of “Bioinformatics” in genetic functional analysis, comprising nothing more than the use of various conventional databases, primer designing programs, primer detection programs, and the like as needed, separately.

To achieve the aforementioned objective, we devised a scheme completely the opposite of conventional methods of genetic analysis. The method of analysis in the present invention is depicted in FIG. 8. That is, in conventional methods, genetic analysis proceeds by a scheme in which the exon which is the target of research is first determined, and primers corresponding to the exon are then designed. In contrast to this, the inventors have devised a scheme in which a plurality of primers are first designed (step 701) for mutually different exons by means of bioinformatics from nucleotide sequence data compiled in public databases or the like (step 700), and DNA fragments which have been amplified by PCR using these primers are then analyzed. This scheme determines which exons are amplified by which primers beforehand (step 702) to make it easier to analyze DNA fragments amplified by PCR, resulting in more efficient analysis. For example, during the analysis of differences in gene levels occurring between normal individuals and patients afflicted with a certain disease (such as cancer), genomic DNAs extracted from the cells of various individuals are used as templates to carry out PCR using a plurality of primers for mutually different exons, and exons which are believed to be related to the disease can be determined based on types of primers having differences in nucleotide sequences and the length or presence/absence of amplified fragments. Thus, in the method of genetic analysis devised by the inventors, PCR is carried out using primers for mutually different exons, and exons compatible with the purpose of research are then determined and analyzed.

In this type of genetic analysis, it is necessary to prepare primers for as many exons as possible. Massive amounts of data have been compiled at present for genomic DNA nucleotide sequences and cDNA nucleotide sequences (see FIG. 1). We have constructed a primer design system in which a computer can be used to process data on DNA nucleotide sequences obtained from databases including a plurality of different DNA nucleotide sequences, so as to design a plurality of primers for mutually different DNAs, and we have also discovered that genetic analysis can be managed more efficiently by correlating the designed primer data and the genetic data of the DNA fragments amplified by PCR using such primers.

The present invention was perfected based on the above findings.

That is, the present invention comprises the following inventions:

(1) a primer design system, comprising: a receiver for obtaining data on a plurality of DNA nucleotide sequences from a first database having data on a plurality of different DNA nucleotide sequences; and a control unit for controlling the system, the aforementioned control unit controlling: extracting means for extracting partial sequences meeting certain base length extraction conditions from the plurality of DNA nucleotide sequences, the data for which were obtained by the aforementioned receiver; detecting means for detecting certain conditions related to the positions of the aforementioned partial sequences, and conditions of their absence in DNA sequences other than the aforementioned DNA nucleotide sequences; first selecting means for selecting partial sequences meeting the aforementioned conditions from the aforementioned partial sequences based on the results of the aforementioned detecting means; and determining means for determining the nucleotide sequence of primers capable of specifically hybridizing to the aforementioned plurality of DNA nucleotide sequences based on the results of the aforementioned first selecting means;

(2) a primer design system according to (1) above, the aforementioned control unit further controls second selecting means for selecting DNA nucleotide sequences meeting certain selection conditions from the partial sequences extracted by the aforementioned extracting means;

(3) a primer design system according to (2) above, the aforementioned selection conditions being related to GC content and/or Tm;

(4) a primer design system according to out of from (1) to (3) above, the aforementioned control unit further controls limiting means for limiting the plurality of DNA nucleotide sequences, the data for which were obtained by the aforementioned receiver, to a base length longer than the aforementioned prescribed base length, to be output to the aforementioned extracting means;

(5) a primer design system according to out of from (1) to (3) above, the aforementioned control unit further controls third selecting means for selecting DNA nucleotide sequences meeting selection conditions related to GC content and/or Tm based on the plurality of DNA nucleotide sequences, the data for which were obtained by the aforementioned receiver;

(6) a primer design system according to out of from (1) to (3) above, further comprising a second database including data for a plurality of different DNA nucleotide sequences, the aforementioned second database comprising at least one of either data on cDNA nucleotide sequences included in the aforementioned first database, or data on the exon nucleotide sequences predicted on the basis of genomic DNA nucleotide sequences included in the aforementioned first database, wherein the aforementioned extracting means targets the aforementioned nucleotide sequences included in the aforementioned second database for extraction;

(7) a storage medium having recorded thereon a program executable at the control unit in a computer having the aforementioned control unit and memory with data on a plurality of different DNA nucleotide sequences, the aforementioned program comprising instruction for reading data on a plurality of DNA nucleotide sequences in the aforementioned memory, for extracting partial sequences having a prescribed base length from the aforementioned nucleotide sequences based on data on the aforementioned read plurality of DNA nucleotide sequences, for detecting certain conditions related to the positions of the aforementioned partial sequences and conditions of their absence in DNA nucleotide sequences other than the aforementioned DNA nucleotide sequences, for selecting partial sequences meeting the aforementioned conditions, and for determining the nucleotide sequences of primers capable of hybridizing specifically to the aforementioned plurality of DNA nucleotide sequences based on the aforementioned selected partial sequences;

(8) a method for designing primers, comprising the steps of: taking data on a plurality of DNA nucleotide sequences from a database including a plurality of different DNA nucleotide sequences; extracting partial sequences having a certain base length from the aforementioned plurality of DNA nucleotide sequences based on the aforementioned nucleotide sequence data obtained above; detecting certain conditions related to the positions of the aforementioned partial sequences, and conditions of their absence in DNA nucleotide sequences other than the aforementioned DNA nucleotide sequences; selecting partial sequences meeting the aforementioned conditions from the aforementioned partial sequences based on the aforementioned detecting results; and determining the nucleotide sequences of primers capable of specifically hybridizing to the aforementioned DNA nucleotide sequences based on the aforementioned selected partial sequences;

(9) a computer-readable storage medium used in bioinformatics, the aforementioned storage medium comprising recorded data on a plurality of primers capable of specifically hybridizing to mutually different DNAs, and genetic data on DNA fragments amplified by PCR using the aforementioned plurality of primers, which are correlated each other;

(10) a computer-readable storage medium comprising data on a plurality of primers capable of specifically hybridizing to mutually different DNAs, and genetic data on DNA fragments amplified by PCR using the aforementioned plurality of primers, which are correlated each other, as well as a recorded program for displaying on a display device genetic data on the aforementioned DNA fragments corresponding to data on the aforementioned plurality of primers input by means of input/output unit of a computer;

(11) a method for analyzing DNA, comprising the analysis of sample DNA using as an indicator the type of primer affording PCR amplified fragments among the aforementioned plurality of primers, using a DNA analysis kit comprising a storage medium according to (9) or (10) above and a plurality of primers, the data for which have been recorded on the aforementioned storage medium;

(12) a DNA analysis kit, comprising a storage medium according to (9) or (10) above, and a plurality of primers for which the aforementioned primer data are recorded;

(13) PCR plates, comprising 75 or more types of solution comprising 1 or more primers;

(14) micro-well plates for PCR, comprising a plurality of solutions comprising 1 or more primers, the primer concentration in the aforementioned solutions ranging between 10 and 100 pmol/μL, with no enzymes that degrade the primers in the aforementioned solutions;

(15) micro-well plates for PCR, comprising a plurality of wells, 80% or more of the total of the aforementioned plurality of wells containing mutually different solutions comprising 1 or more primers;

(16) micro-well plates for PCR according to out of from (13) to (15) above, comprising the plurality of primers designed by means of a primer design method comprising the steps of: taking data on a plurality of DNA nucleotide sequences from a database including a plurality of different DNA nucleotide sequences; limiting the base length of the aforementioned plurality of DNA nucleotide sequences to a certain base length based on the aforementioned nucleotide sequence data taken above; extracting first partial sequences having a certain base length from the aforementioned limited nucleotide sequences; selecting second partial sequences meeting selection conditions related to GC content and/or Tm from the aforementioned first partial sequences; detecting certain conditions related to the positions of the aforementioned second partial sequences, and conditions of their absence in DNA nucleotide sequences other than the aforementioned DNA nucleotide sequences; selecting third partial sequences meeting the aforementioned conditions from the aforementioned second partial sequences based on the aforementioned detected results; and determining the nucleotide sequence of primers capable of specifically hybridizing to the aforementioned DNA nucleotide sequences based on the aforementioned third partial sequences;

(17) micro-well plates for PCR according to out of from (13) to (15) above, comprising a plurality of primers designed by means of a primer design method comprising the steps of: taking data on a plurality of DNA nucleotide sequences from a database including a plurality of different DNA nucleotide sequences; selecting DNA nucleotide sequences meeting selection conditions related to GC content and/or Tm from a plurality of DNA nucleotide sequences, the data for which have been obtained above; extracting partial sequences having a certain base length from the aforementioned selected nucleotide sequences; detecting certain conditions related to the positions of the aforementioned partial sequences, and conditions of their absence in DNA nucleotide sequences other than the aforementioned DNA nucleotide sequences; selecting partial sequences meeting certain conditions from the aforementioned partial sequences based on the aforementioned detected results; and determining the nucleotide sequence of primers capable of specifically hybridizing to the aforementioned DNA nucleotide sequences based on the aforementioned selected partial sequences;

(18) a PCR amplifying kit comprising a plurality of primers and a computer-readable storage medium, the aforementioned PCR amplifying kit comprising containers containing the aforementioned plurality of primers, ID codes assigned to the primers contained in the containers being indicated on the aforementioned containers, and a table correlating the aforementioned ID codes of the aforementioned plurality of primers with either the name, molecular formula, or sequence data for the aforementioned plurality of primers being recorded on the aforementioned storage medium.

### BRIEF DESCRIPTION OF THE DRAWINGS

The foregoing and additional features and characteristics of the present invention will become more apparent from the following detailed description considered with reference to the accompanying drawings in which like reference numerals designate like elements and wherein:

FIG. 1 illustrates changes in the number of nucleotide sequences registered at GenBank;

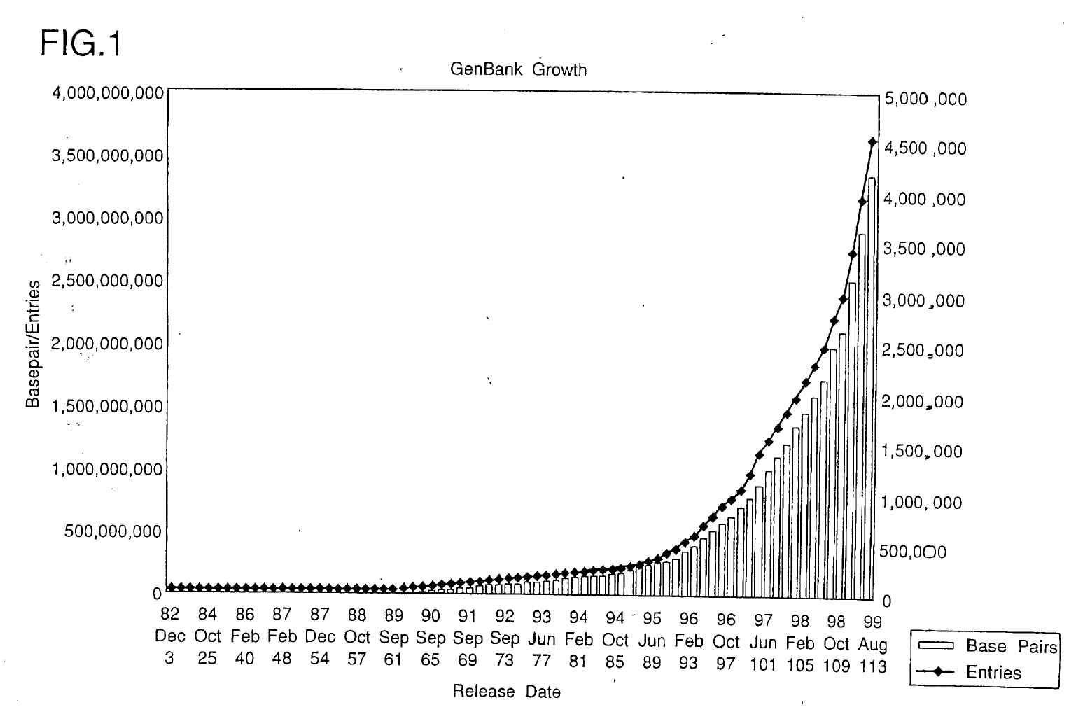

FIG. 2 is a block diagram illustrating an example of the structure of the primer design system in the present invention;

FIG. 3 is a flow chart illustrating the construction of a database using a public database;

FIG. 4 is a block diagram illustrating an example of the structure of a primer designing program;

FIG. 5 is a flow chart illustrating an example of a process using the program illustrated in FIG. 4;

FIG. 6 illustrates exon sequences of sequences selected from the sequence database for chromosome 21, and partial sequences extracted under certain extraction conditions from these exon sequences;

FIG. 7 illustrates a conventional method of DNA analysis;

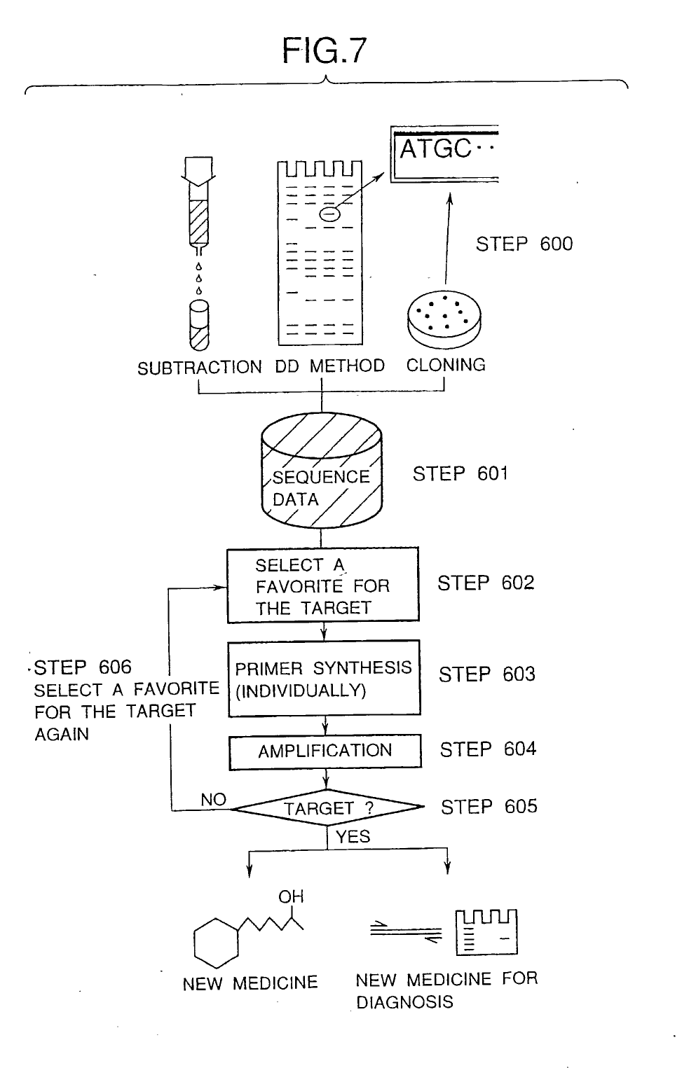

FIG. 8 illustrates the method of DNA analysis in the present invention;

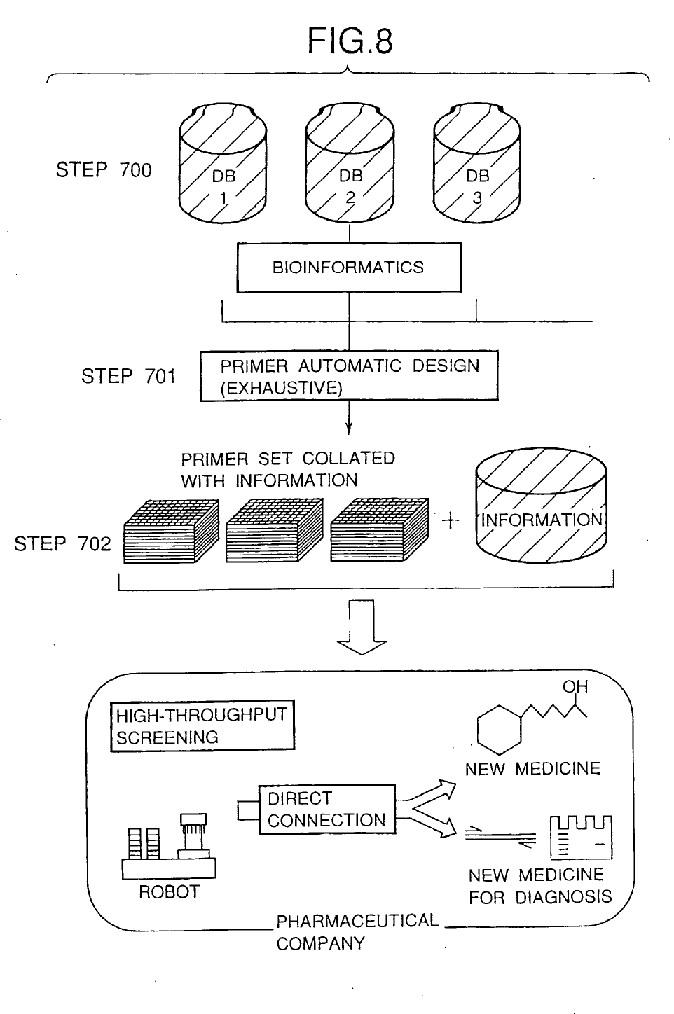

FIG. 9 shows the detailed flow chart of a primer design system according to the present invention;

FIG. 10 shows the flow chart of the primer design data conversion in step 1500 of FIG. 9;

FIG. 11 shows the flow chart of the primer evaluation data conversion in step 1700 of FIG. 9;

FIG. 12 depicts an example of the exon predictive data conversion stage 1 in step 1100 of FIG. 9;

FIG. 13 depicts an example of the exon predictive data conversion stage 2 in step 1200 of FIG. 9;

FIG. 14 depicts an example of converting the primer data based upon the exon prediction data;

FIG. 15 depicts an example of converting the primer data based upon the I/E junction data;

FIG. 16 shows the results of the primer design data conversion in step 1500 of FIG. 9;

FIG. 17 depicts the data processed by Primer0.5;

FIG. 18 contracts the conventional system and the primer-design system according to the invention;

FIG. 19 shows plural primer design systems simultaneously designing primers from plural genomic DNA sequences;

FIG. 20 shows plural primers are designed simultaneously based upon not plural exons according to the invention;

FIG. 21 shows each function being processed by independent CPUs to further improve efficiency; and

FIG. 22 depicts the specificity evaluation for exons and primers.

### DESCRIPTION OF THE PREFERRED EMBODIMENTS

The present invention is described in detail below.

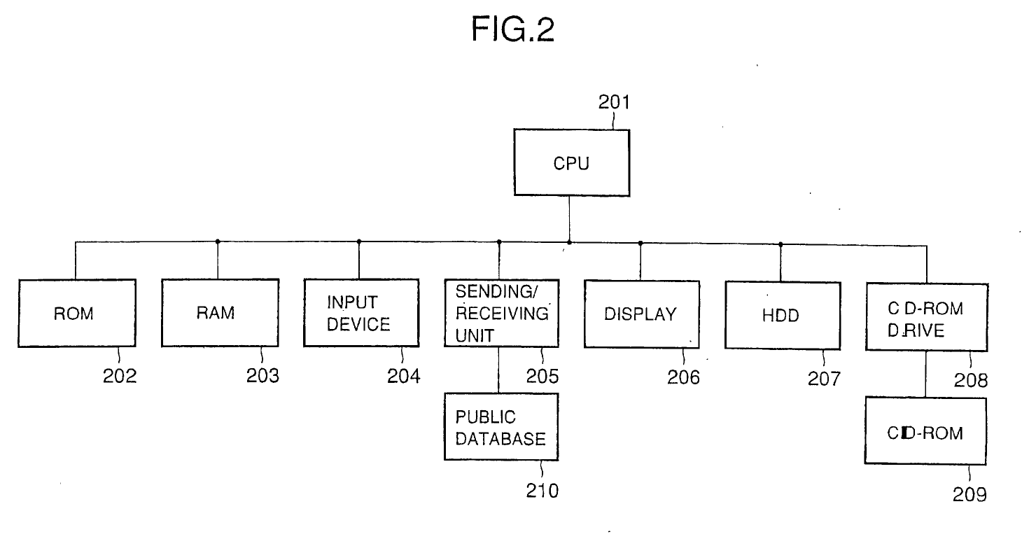

FIG. 2 is a block diagram illustrating an example of the structure of the primer design system in the present invention. The primer design system illustrated in FIG. 2 comprises CPU 201, ROM 202, RAM 203, input 204, transmitter/receiver 205, display 206, hard disc drive (HDD) 207, and CD-ROM drive 208. A re-writable CD-R or CD-RW can be used as storage medium instead of the CD-ROM 209. In such cases, CD-R or CD-RW drive is used instead of the CD-ROM drive 208. DVD, ZiP, MO, PD and corresponding drives for such media may also be used as the media for storing the large volume of primer-related data instead of the CD-ROM 209.

The CPU 201 runs the primer designing process described below and controls the primer design system as a whole according to programs stored on the ROM 202, RAM 203, or hard disc drive (HDD) 207. ROM 202 stores the programs or the like giving commands for the process needed to operate the primer design system. RAM 203 temporarily stores data necessary for running the primer design process. The input 204 is a keyboard, mouse, or the like, and is used to input the necessary conditions for running the primer design process. The transmitter/receiver 205 transfers data to and from public databases 210 or the like through communication lines based on commands from the CPU 201. The display 206 displays DNA nucleotide sequences obtained from databases, various conditions input from the input 204, designed primer nucleotide sequences, and the like based on commands from the CPU 201. The hard disc drive (HDD) 207 stores databases and the like comprising a plurality of different DNA nucleotide sequences and the primer design program, reads the stored programs, data, and the like based on commands from the CPU 201, and stores them in RAM 203, for example. The CD-ROM drive 208 reads programs, data, and the like from databases comprising a plurality of different DNA nucleotide sequences and the primer design program stored in the CD-ROM 209 based on commands from the CPU 201, and stores them in RAM 203, for example.

In the primer design system of the present invention, the receiver receives DNA nucleotide sequences from a database comprising a plurality of different DNA nucleotide sequences.

In the primer design system illustrated in FIG. 2, DNA nucleotide sequences contained in a public database 210 (a first database), for example, can be received by the transmitter/receiver 205 through communications lines, and these DNA nucleotide sequences can be stored in RAM 203. Specific examples of a public database 210 include databases which can be used over the Internet (WWW (world wide web)). More specific examples include GenBank (nucleic acid nucleotide sequence (including DDBJ) database, prepared by NCBI (USA), National Genetic Research Institute), EMBL (nucleic acid nucleotide sequence database, prepared by EBI (Europe)), nr-nt (nucleic acid nucleotide sequence database, prepared from GenBank and EMBL), GENOME (KEGG genome maps, prepared by Kyoto University Chemical Research Institute), GENES (KEGG gene catalogs, prepared by Kyoto University Chemical Research Institute), CHR21 (sequence map for chromosome 21, prepared by HGC), JST (JST human genome sequencing database, prepared by Japan Science and Technology Corporation), BodyMap (human gene expression database, prepared by Osaka University), GENOTK (human cDNA database, prepared by Otsuka Pharmaceutical Co. Ltd., HGC), and MBGD (microorganism genome database, prepared by HGC). Nucleotide sequences received from a public database 210 may be either cDNA nucleotide sequences or genomic DNA nucleotide sequences, or partial sequences thereof. When the nucleotide sequences obtained from a public database 210 are cDNA nucleotide sequences, the cDNA nucleotide sequences received by the transmitter/receiver 205 are stored without modification in RAM 203. When the nucleotide sequences obtained from a public database 210 are genomic DNA nucleotide sequences, the genomic DNA nucleotide sequences are processed by an exon predicting program stored in ROM 202, hard disc drive (HDD) 207, or CD-ROM 209 which predicts the exon nucleotide sequences based on the genomic DNA nucleotide sequences, and the predicted exon nucleotide sequences are then stored in RAM 203. Existing exon predicting programs such as GENSCAN, GRAIL, and ER (Exon Recognizer) can be used as the exon predicting program.

In the primer design system illustrated in FIG. 2, DNA nucleotide sequences included in a database stored, for example, in the hard disc drive (HDD) 207 or CD-ROM 209 can be read based on commands from the CPU 201 and stored in RAM 203. A specific example of a database stored in the hard disc drive 207 or CD-ROM 209 is a locally built database using a public database.

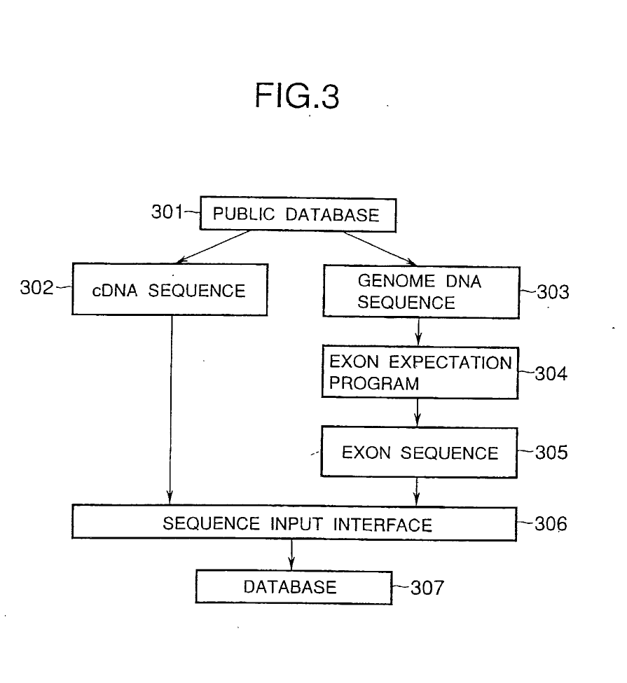

FIG. 3 is a flow chart illustrating the construction of a database using a public database.

cDNA sequences 302 included in a public database 301 (a first database), and exon sequences 305 obtained when genomic DNA sequences 303 included in a public database 301 are processed by the exon predicting program 304, can be stored in the hard disc drive or other recordable storage medium through a sequence input interface 306, so as to construct a database 307 (a second database). When constructing the database, the cDNA nucleotide sequences or exon nucleotide sequences can be divided to suitable lengths (such as 1 kb) and stored in a storage medium. Existing exon predicting programs such as GENSCAN, GRAIL, and ER (Exon Recognizer) can be used as the exon predicting program, and these programs can be used over the Internet. The database 307 built in this manner contains a plurality of different DNA nucleotide sequences.

The CPU 201 supplies the DNA nucleotide sequences received from the database to the display 206, and runs the process for designing primers capable of hybridizing specifically to the DNA received from the database (hereinafter referred to as “primer design process”). In the primer design system of the present invention, after the DNA nucleotide sequences have been received by the receiver, the primer design process is run by fragment length limiting process, partial sequence extracting process, partial sequence detecting process, partial sequence selecting process, and primer sequence determining process.

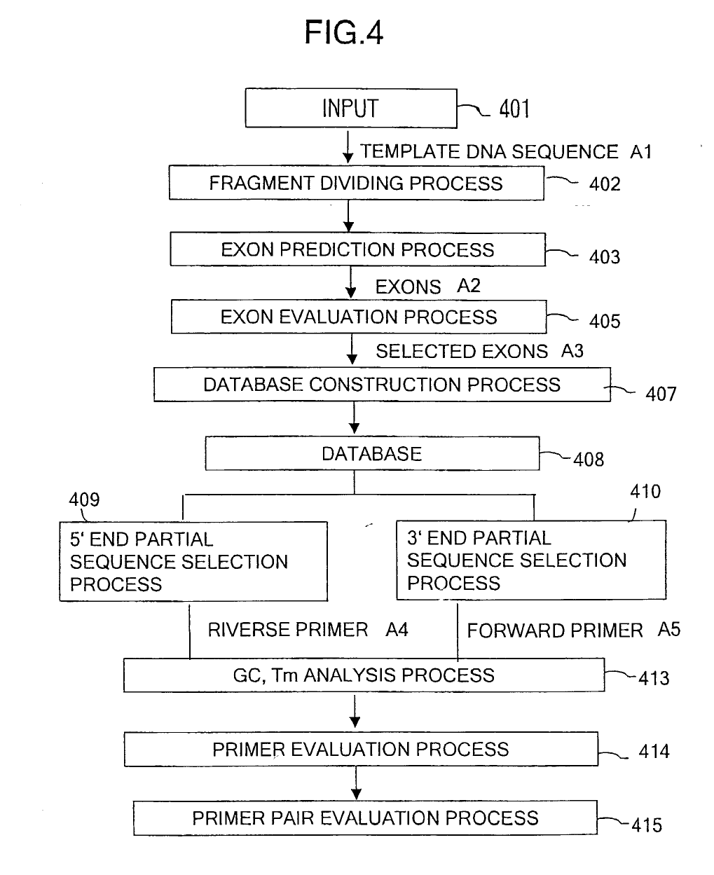

FIG. 4 is a block diagram illustrating an example of the structure of a primer designing program.

The CPU 201 supplies the DNA nucleotide sequences received from the database to the input 401 The input 401 supplies a DNA sequence A1 viaa fragment dividing process 402. The fragment dividing process 402 randomly divides the DNA sequence A1 into a plurality of fragments, and then supplies them to an exon prediction process 403 to predict exons simultaneously. A plurality of exons are predicted from the fragments by the exon prediction process 403, and the exons A2 are supplied to the exon evaluation process 405. The exon evaluation process 405 determines whether or not each of the exons A2 meets certain evaluation conditions to be discussed in details later. [\mathring{1} A specific GC content: the proportion between the sum of cytosine and guanine content and the sum of adenine and thymine content in double-stranded DNA molecules; or Tm: the temperature at which the double-stranded portion of DNA or RNA molecules is denatured into single strands, resulting in a double-stranded/single-stranded ratio of 1:1]]]]]]]]]]]]]]]].] The evaluation conditions can be selected as desired. [\mathring{2} Specific examples of such detection conditions include conditions under which the GC content is 50 to 60%, the Tm is between 50 and 80° C., and |ΔTm| is below 20° C. (i.e. the pairs of primers have their annealing temperatures ±20° C.)] The exon evaluation process 405 supplies the selected exons A3 meeting the prescribed evaluation conditions to a database constructing process 407 (a second selecting means).

The database construction process 407 constructs a database 408 comprising the selected exons A3 meeting the prescribed evaluation conditions. From the exons contained in the database 408, a 5′ partial sequence selection process 409 selects partial sequences located closest to the 5′ end among the exons derived from the DNA sequence A1. From the exons contained in the database 408, a 3′ partial sequence selection process 410 selects partial sequences located closest to the 3′ end among the exons derived from the DNA sequence A1. The partial sequences A4 selected by the 5′ primer selection process 409 and the partial sequence A5 selected by the 3′ partial sequence selection process 410 are supplied to a GC, Tm analysis process 413. The GC, Tm analysis process 413 analyzes whether or not the supplied primers A4 or A5 satisfy \mathring{1},\mathring{2}. To determine whether or not the supplied primers are present in DNA nucleotide sequences other than the template DNA, a primer evaluation process 414 analyzes data compiled in public databases and the like by means of a homology screening program. BLAST or FASTA, for example, can be used as the homology screening program. The primer evaluation process 414 selects primers that are not present in DNA nucleotide sequences other than the template DNA and supplies the selected primers A6 (a third partial sequences) to a primer pair evaluation process 415 . The primer pair evaluation process 415 determines the specificity of primer pairs on the DNA sequence. [\mathring{3} The nucleotide sequence that is complementary to the partial sequence of the 5′ end is supplied as a forward primer, and the nucleotide sequence that is complementary to the partial sequence of the 3′ end is supplied as a reverse primer.] Primers capable of hybridizing specifically to the exons can be designed in this manner.

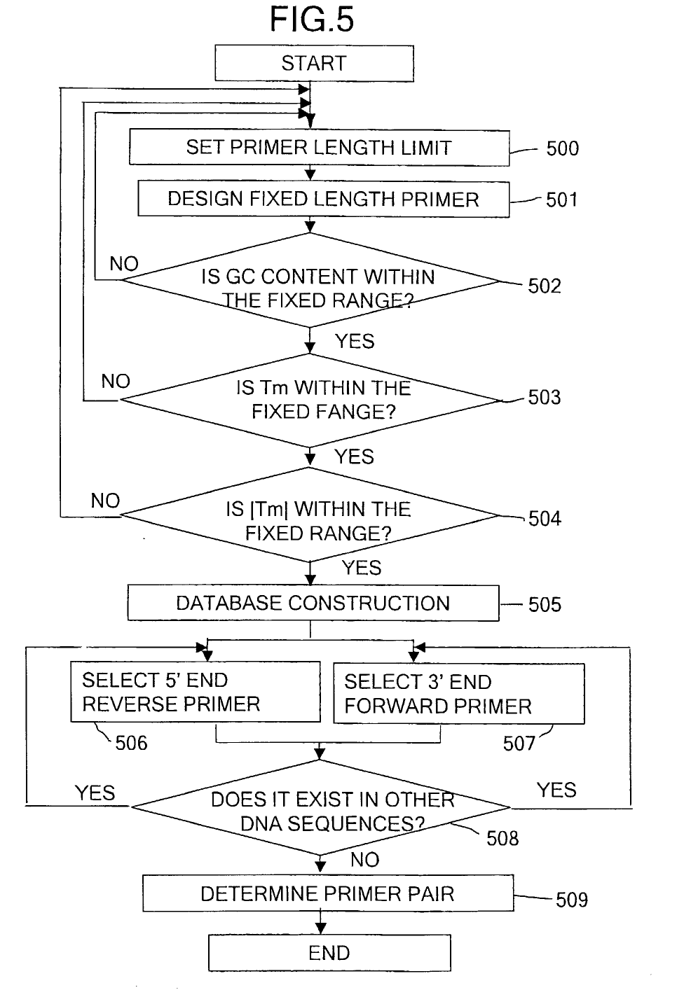

FIG. 5 is a flow chart illustrating the handling of primers in FIG. 4 in detail.

A partial sequence of prescribed base length (such as 20 to 28 bases) are selectedin a step 501 from the predicted exons. The GC, Tm analysis process 413 determines whether or not the GC content of the extracted partial sequence is within a prescribed range (such as 50 to 60%) (step 502). When the GC content of the extracted partial sequence is not within the prescribed range (such as 50 to 60%) they are discarded. When the GC content of the extracted partial sequence is within the prescribed range (such as 50 to 60%), it is then determined whether or not the Tm is within a prescribed range (such as 50 to 80° C.) (step 503). When the Tm of the extracted partial sequence A2 is not within the prescribed range (such as 50 to 80° C.), they are discarded. When the Tm is within the prescribed range (such as 50 to 80° C.), it is then determined whether or not |ΔTm| is within a prescribed range (such as below 20° C.) (step 504). When the |ΔTm| of the extracted partial sequence is not within the prescribed range (such as smaller than 20° C.), they are discarded. When the |ΔTm| is within the prescribed range (such as smaller than 20° C.), the partial sequence is recorded in a re-writable storage medium such as the hard disc or CD-R by a database construction process. Steps 501 through 505 are repeated for all partial sequences that are designed from the exons to construct a database of partial sequences meeting the prescribed extraction conditions (such as a base length of 20 to 28 bases, a GC content of between 50 and 60%, a Tm of between 50 and 80° C., and a |ΔTm|smaller than 20° C.) (step 505). From the partial sequences contained in the database that has been constructed, the 5′ partial sequence selection process 409 selects partial sequences located closest to the 5′ end (step 506). In addition, from the partial sequences contained in the database that has been constructed, the 3′ partial sequence selection process 410 selects partial sequences located closest to the 3′ end (step 507). The partial sequence A4 selected by the 5′ partial sequence selection process 409 and the partial sequence A5 selected by the 3′ partial sequence selection process 410 are analyzed by the primer evaluation process 414 to determine whether or not they are present in DNA nucleotide sequences other than the template DNA (step 508). When the partial sequence A4 selected by the 5′ partial sequence selection process 409 is present in a DNA nucleotide sequence other than the template DNA, the partial sequence located second closest to the 5′ end is then selected from the partial sequences contained in the database that has been constructed (step 506). When the partial sequence A5 selected by the 3′ partial sequence selection process 410 is present in a DNA nucleotide sequence other than the template DNA, the partial sequence located second closest to the 3′ end is then selected from the partial sequences contained in the database that has been built (step 507). Steps 506 through 508 are repeated until a partial sequence that is not present in a DNA nucleotide sequence other than the template DNA is selected. Partial sequences that are not present in DNA nucleotide sequences other than the template DNA are selected by the partial sequence evaluation process 414, and the selected partial sequences A6 are supplied to the primer pair evaluation process 415. Primers capable of hybridizing specifically to the template exons are designed by the primer pair evaluation process 415 based on partial sequences that are not present in DNA nucleotide sequences other than the template DNA (step 509).

The primers designed by means of the primer design system of the present invention can be chemically synthesized by a common method according to their nucleotide sequences. The primer design system of the present invention makes it possible to efficiently design a plurality of primers capable of hybridizing the exons.

In the present embodiment, partial sequences closest to the 5′ or 3′ end were selected after detection of Tm or the like, and sequences analyzed as not being included anywhere except in the template DNA were determined as primer sequences, but the order of the detection, selection, and analysis may be changed. When exons in their entirety are to be analyzed, or when exon-intron junctions are to be analyzed, the object of primer design is not limited to exon regions, and the partial sequences for introns can also be used for template DNAs.

A plurality of primers capable of specifically hybridizing the exons can be used in DNA analysis.

For example, a sample DNA can be used as a template, PCR can be run using a plurality of primers capable of specifically hybridizing the exons, and the sample DNA can be analyzed using the types of primers giving the PCR amplified fragments as markers. For example, during the analysis of differences in gene levels between normal individuals and patients afflicted with a certain disease (such as cancer), genomic DNA extracted from the cells of individuals can be used as templates, PCR can be run using a plurality of primers capable of hybridizing specifically to exons such that exons potentially related to the disease can be determined based on types of primers having differences in nucleotide sequence and the length or presence/absence of amplified exons between normal individuals and patients. High-throughput screening is made possible by DNA analysis thus using a plurality of primers capable of hybridizing specifically to the exons.

In DNA analysis featuring the use of a plurality of primers capable of hybridizing specifically to exons, it is important to collate the data of the primers with the genetic data of the DNA fragments (i.e. exons) amplified by PCR using the primers. Specifically, it is important to determine the genetic data of the DNA fragments amplified using the primers based on the data of the primers affording the fragments amplified by PCR. It is thus desirable to use a computer-readable storage medium to record the data of the plurality of primers capable of hybridizing exons, and the genetic data of the DNA fragments amplified by PCR using these primers. A program for allowing the display of the genetic data of the DNA fragments amplified by PCR using these primers based on the data of the primers input to a computer may be recorded in the storage medium. The program may also be recorded in another storage medium.

The primer data include primer nucleotide sequences, data characterizing the primer (such as identifying name), or the like. The genetic data of the DNA fragments include DNA fragment nucleotide sequences, data related to the function of the proteins encoded by the DNA fragments (whether or not functions have been elucidated, and which functions have been elucidated), or the like. Storage media include CD-ROM, hard disc, ROM, RAM, DVD, and CD-R/RW.

The aforementioned DNA analysis can be performed using a DNA analysis kit comprising a plurality of primers capable of hybridizing specifically to mutually different DNAs, and the aforementioned storage medium. A PCR amplifying kit comprising a plurality of primers and a computer-readable storage medium can be used in the aforementioned DNA analysis. Each of the aforementioned plurality of primers is contained in a plurality of containers in such a PCR amplifying kit, ID codes given to the primers contained in the containers are indicated on the aforementioned plurality of containers, and a table collating the ID codes of the aforementioned plurality of primers with either the name, molecular formula, or sequence data for the aforementioned plurality of primers is recorded in the aforementioned storage medium. Plates having a plurality of wells as described below can be used as the containers.

DNA can be analyzed using the aforementioned DNA analysis kit in the following manner, for example. An identification name (ID code) such as B1, B2, B3 through Z7, Z8, Z9, for example, is given to each primer as data characterizing the primers, and “B5” is input as primer data to the input 204 when the primer giving PCR amplified fragments is B5 during PCR run with the primers. The CPU 201 determines the genetic data of the DNA fragments which have been amplified by PCR using primer B5 based on the input primer data in accordance with the program stored in ROM 202, RAM 203, hard disc 207, or CD-ROM 209, and displays on the display 206.

For efficient analysis of large amounts of sample DNA during DNA analysis, it is possible to use plates having a plurality of wells, which are plates containing in some of the wells solutions containing the plurality of primers capable of hybridizing specifically to mutually different DNAs. Such plates can be used to carry out PCR all at once using a plurality of primers for sample DNAs, thus allowing the sample DNAs to be efficiently analyzed and large amounts of sample DNA to be analyzed. PCR featuring the use of such plates can be carried out with commercially available automated devices such as automatic reaction robots.

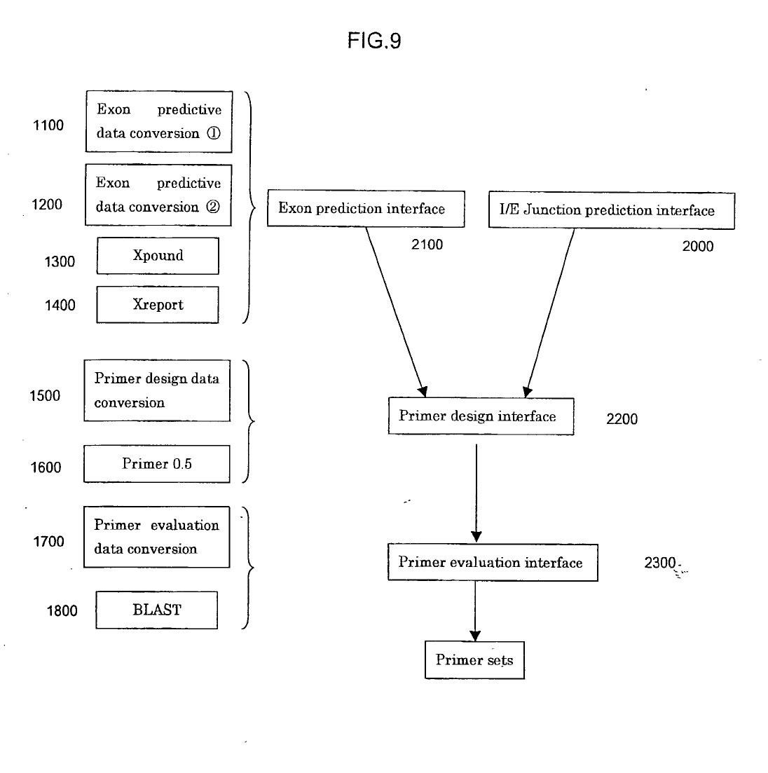

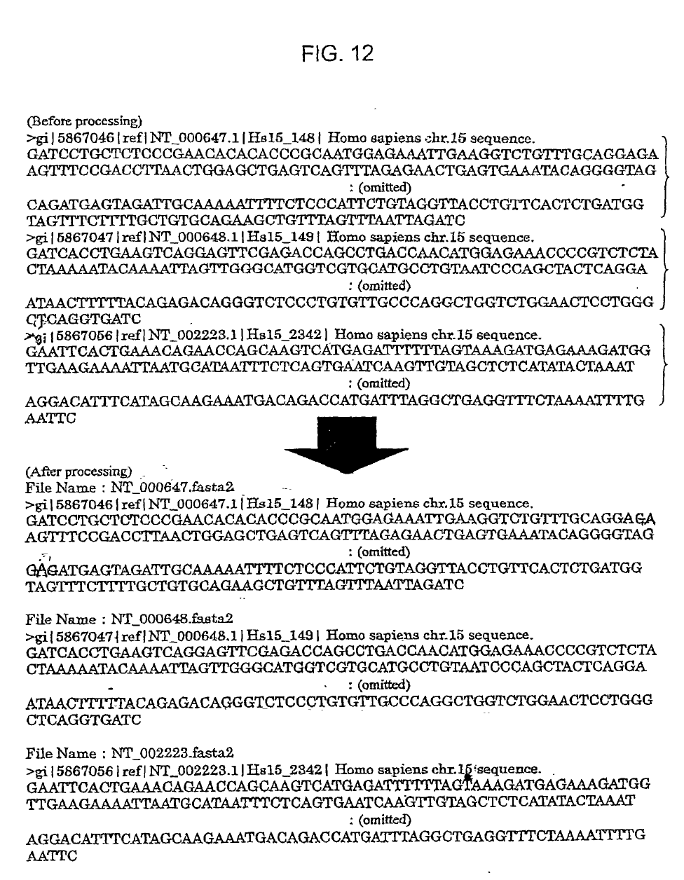

A primer design system according to the present invention proceeds according to the detailed flow chart depicted in FIG. 9. In the Exon predictive data conversion stage 1 (step 1100), each of a plurality of genomic sequences is randomly divided into, for example, three fragments and saved in three separate files. An example of the Exon predictive data conversion stage 1 is shown in FIG. 12.

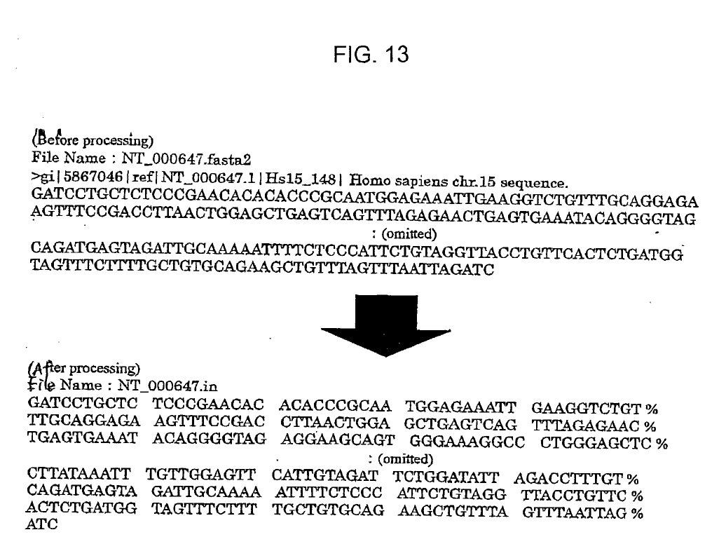

In the Exon predictive data conversion stage 2 (step 1200), one blank is inserted into every 10 characters in each of the three sequences, and a carriage runs every 50 characters according to the Xpound (step 1300) input format. An example of the Exon predictive data conversion stage 2 is shown in FIG. 13. In short, a plurality of exons are simultaneously predicted via steps 1100-1300 by taking each of the plurality of fragments as a template.

Xpound is a publicly available software for exon trapping. Exon trapping (exon prediction) is a rapid and efficient means for finding expressed DNA sequences in a genome sequence and is based on selecting functional splice sites in the nucleotide sequences of genomic DNAs. The advantages of exon trapping are that it does not require any prior knowledge about gene expression and can easily be performed on complex genomes including polymorphism. Xpound can identify constitutive exons as well as alternative exons but cannot be used to identify intronless genes, such as bacterium genomes which do not have any intron. Then the Xpounded data is sent to Xreport (step 1400) for reporting regions of bases for which the probability of coding is high (Exon prediction). The steps 1100-1400 are handled by an Exon prediction interface 2100.

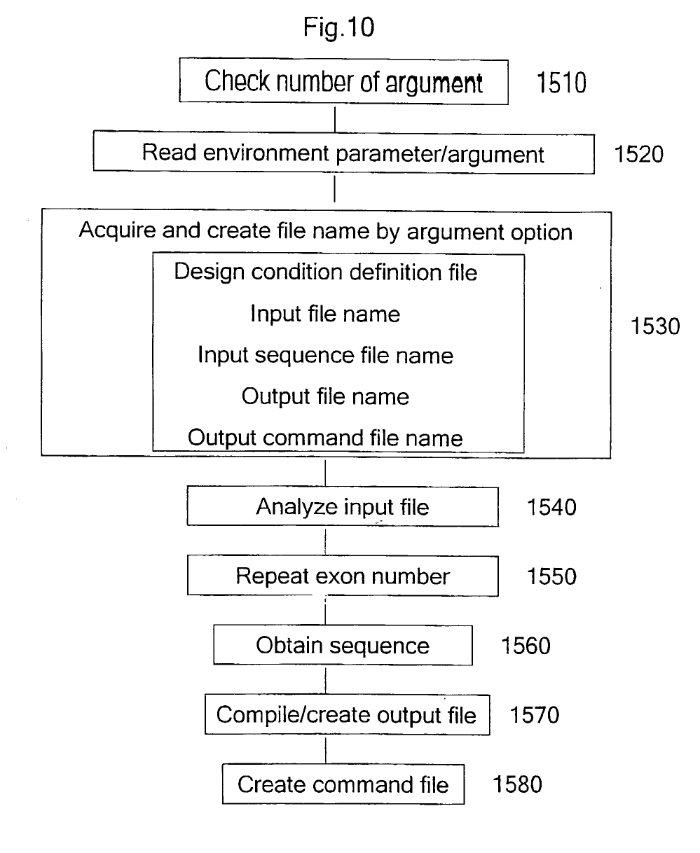

Thereafter, the Xreported data, i.e., the output from Exon prediction interface 2100, or the I/E junction data, are converted into a format readable by Primer0.5 or other computer programs for automatically extracting and selecting PCR primers according to the flow chart depicted in FIG. 10 (step 1500). Alternatively, the data may be manually input via the conventional interactive-base, such as copying and pasting, or creating intermediate files, thereby processing Primer 0.5, and then converted to be executed in batch processing.

In the primer design interface 2200, a plurality of primer pairs are simultaneously designed by using each of the predicted exons as a template. The system first determines whether the input data come from the Exon prediction interface 2100 or they are the I/E junction data. If the data come from the Exon prediction interface 2100, the processing continues from the right side stream of FIG. 9. If it is the I/E junction data, the processing continues from the left side stream of FIG. 9.

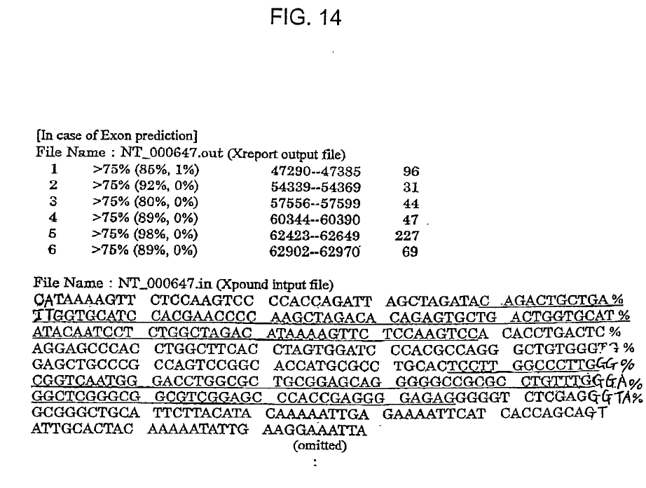

For the output from the Exon prediction interface, the system first checks the number of parameters/arguments in the output from the Exon prediction interface 2100 (step 1510). Then the system reads the relevant environment arguments (step 1520). In step 1530, the system acquires/creates the relevant file names of the sequence data based upon the argument options. The created file includes the information of at least one design condition definition, an input file name, an input sequence file name, an output file name, and an output commend file name. Thereafter, the system analyzes the input file in step 1540 and repeats the exon number in step 1550 so as to acquire the sequences in step 1560. Then the output file is compiled and created in step 1570 so as to generate an output commend file in step 1580. An example of the primer data conversion based upon the output from the Exon prediction interface 2100 is shown in FIG. 14. As shown in FIG. 14, the probability of coding (shown in the second column of the Xreport output file), the positional information of the portions to be amplified, i.e., exons, (sown in the third column of the Xreport output file), and the information of the nucleotide sequences (i.e., the Xpound input file), are separately arranged. Based upon the Xpound specification or the Xreported results, the nucleotide sequences will be extracted based upon the exon positional information, and yet converted into a format readable by Primer0.5.

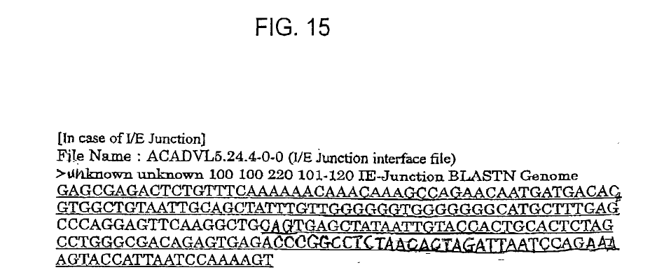

The system also processes I/E junction data obtained from an external source. The techniques of identifying I/E junctions are disclosed, for example, in the article titled “Identification of Coding Regions in Genomic DNA” by Snyder, E. E., Stormo, G. D., J. Mol. Biol. 248: 1-18 (1995). An example of converting the primer data based upon the I/E junction data is shown in FIG. 15. The exon positional information and the information of the nucleotide sequences are bundled in the I/E junction data to be converted into a format readable by Primer0.5.

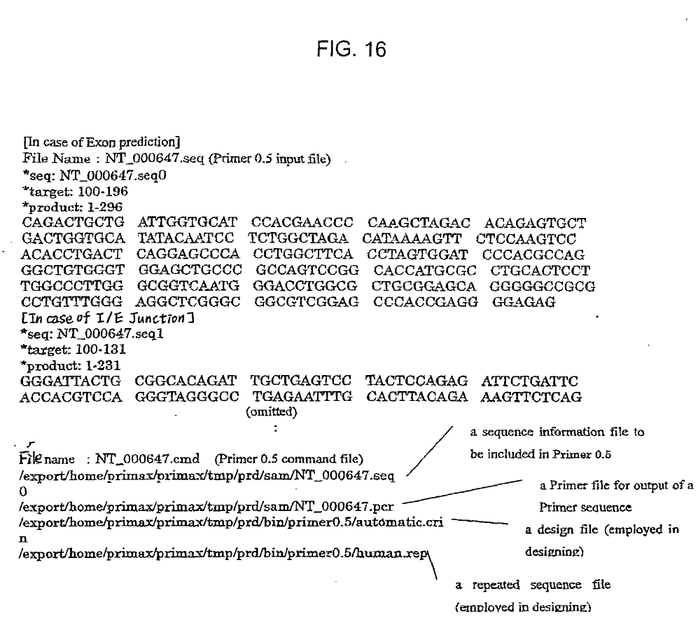

The results of the Primer design data conversion in step 1500 is shown in FIG. 16. The Primer0.5 commend file includes a sequence information file to be included in Primer0.5, a Primer file for outputting a Primer sequence, a design file to be employed in designing the primer pairs, and a duplicate of the sequence information file to be employed in designing the primer pairs. The design is carried out with the Primer0.5 commend file in batch processing.

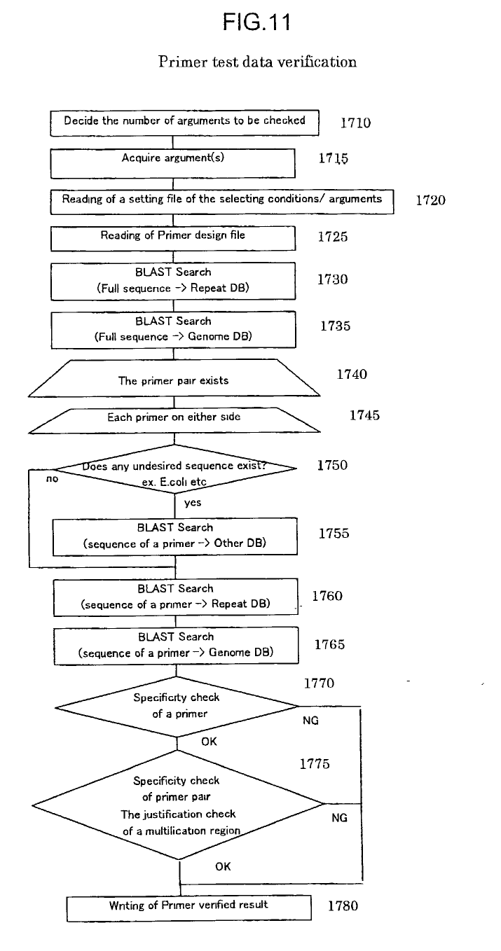

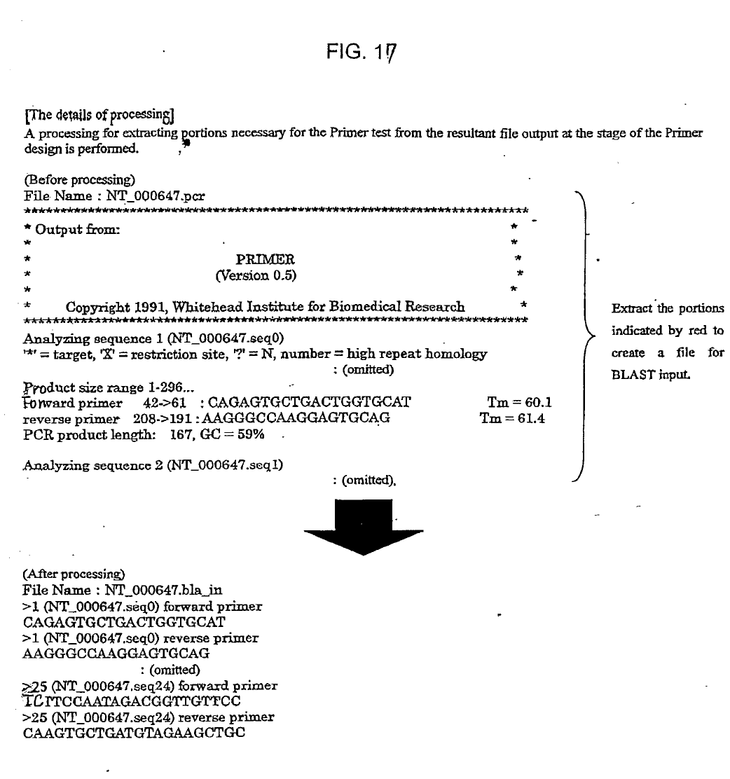

Referring back to FIG. 9, the selected primers by the Primer0.5, i.e., the output from the primer design interface 2200, is then extracted into a format readable by BLAST in step 1700. The data processed by Primer0.5 is shown in the upper portion of FIG. 17. Each analyzed sequence information item includes a product size range, a forward primer with its Tm value, a reverse primer with its Tm value, a PCR product length, and a GC content. The analyzed sequence information item is input into the system in step 1710 according to the flow chart in FIG. 11.

The portions necessary for primer evaluation (shown in the lower portion of FIG. 17) are extracted and compiled from the output of the Primer0.5 in three steps according to FIG. 11. If a sequence is determined as a forward or reverse primer, the relevant file name and sequence are compiled and output. Otherwise, the sequence is searched for forward primers so as to compile and output the searched forward primers. If the sequence is searched as containing no forward primer, it is then searched to see whether it contains any reverse primers. If so, the reverse primers are compiled and output. Thereafter, the output of the primer evaluation data conversion step 1700 is generated which includes a file name, forward primers, and reverse primers as shown in the lower portion of FIG. 17.

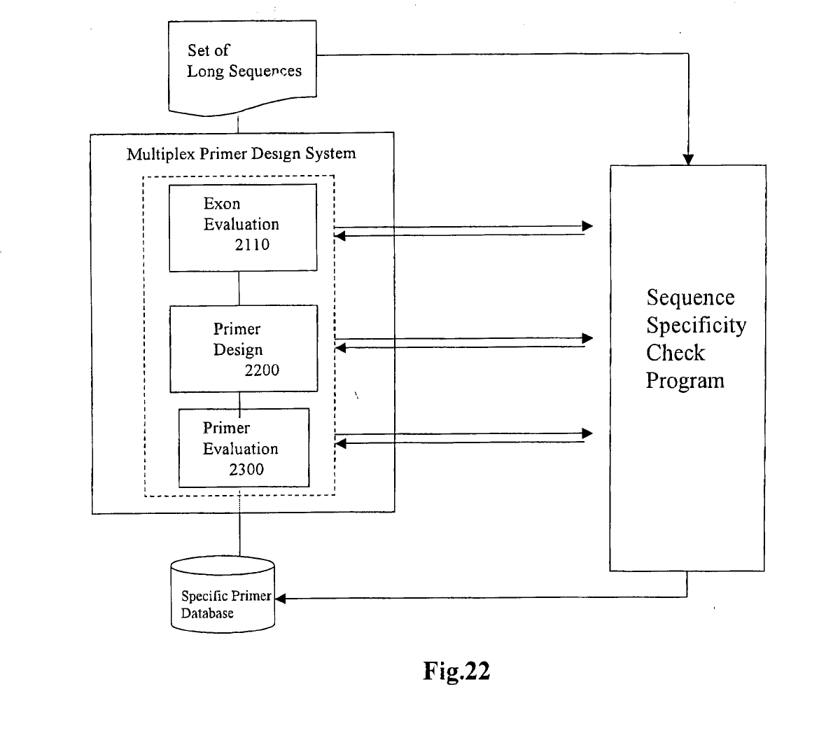

The Basic Local Alignment Search Tool under the trademark BLAST® (hereinafter “BLAST”) is a set of similarity search programs designed to explore all of the available sequence databases regardless of whether the query is protein or DNA. The BLAST programs have been designed for speed, with a minimal sacrifice of sensitivity to distant sequence relationships. As shown in FIG. 22, the exons are evaluated for specificity in the exon prediction interface 2100, and the designed primers are evaluated for specificity in the primer evaluation interface 2300. The scores assigned in a BLAST® search have a well-defined statistical interpretation which makes real matches easier so as to distinguish them from random background hits. The BLAST® tool uses a heuristic algorithm which seeks local as opposed to global alignments and is therefore able to detect relationships among sequences which share only isolated regions of similarity. The system supports different ways to run BLAST®, such as through the Web of the BLAST® network, or to run locally to search against private/local databases.

The present invention not only automates the primer selecting and designing process with the above-mentioned linking steps, such as the exon predictive data conversion steps 1100, 1200, the primer design data conversion step 1500, an the primer evaluation data conversion step 1700, but also processes the operations for plural genomic sequences and plural exons in parallel.

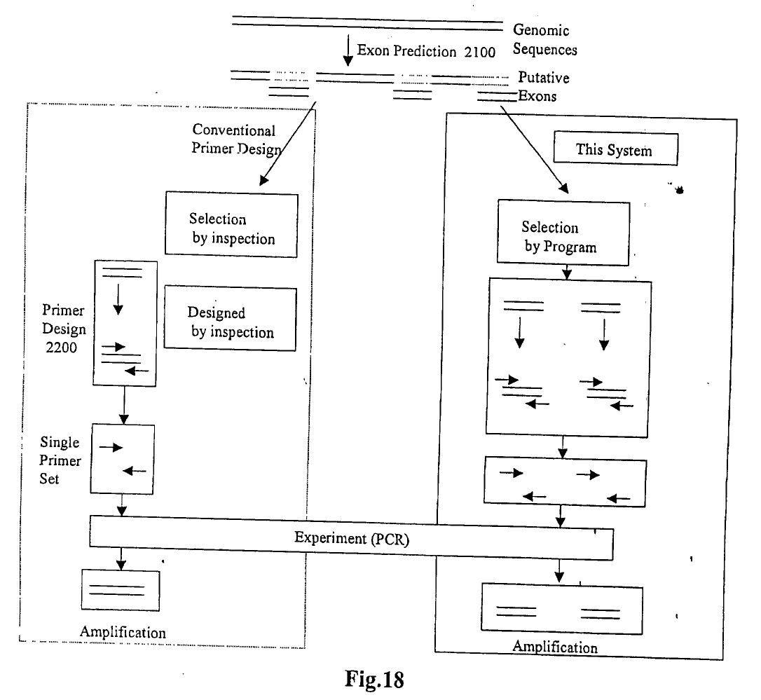

FIG. 18 shows the comparison between the conventional system and this primer-design system in designing primers based upon putative exons deduced from genomic sequences. By the conventional primer design system (left side of FIG. 18), primer design is performed one by one based on the each exon sequence extracted from the genomic sequences. Normally, this process is done by checking the evaluation score of the candidates of the appropriate primer pair with the interactive-type primer design software tools under the trademarks Oligo® or GCG®. Each primer pair consists of two short DNA sequences representing forward and reverse (or 5′ and 3′) primers to amplify object sequences since these combinations are indispensable for the PCR amplification. In this case, only single primer pair is designed in one process after the user checks the score of the primer candidates. Despite that each primer pair requires two short sequences for the amplification, the target region for the PCR is limited to one specific region. Therefore, this conventional primer design system is practically a single-primer design system.

On the other hand, the primer design system of this invention designs plural primer pairs in parallel (right side of FIG. 18). Following the exon prediction, the extracted exon sequences are proceeded for fragment evaluation and primer design so as to automatically obtain the appropriate primer sequences.

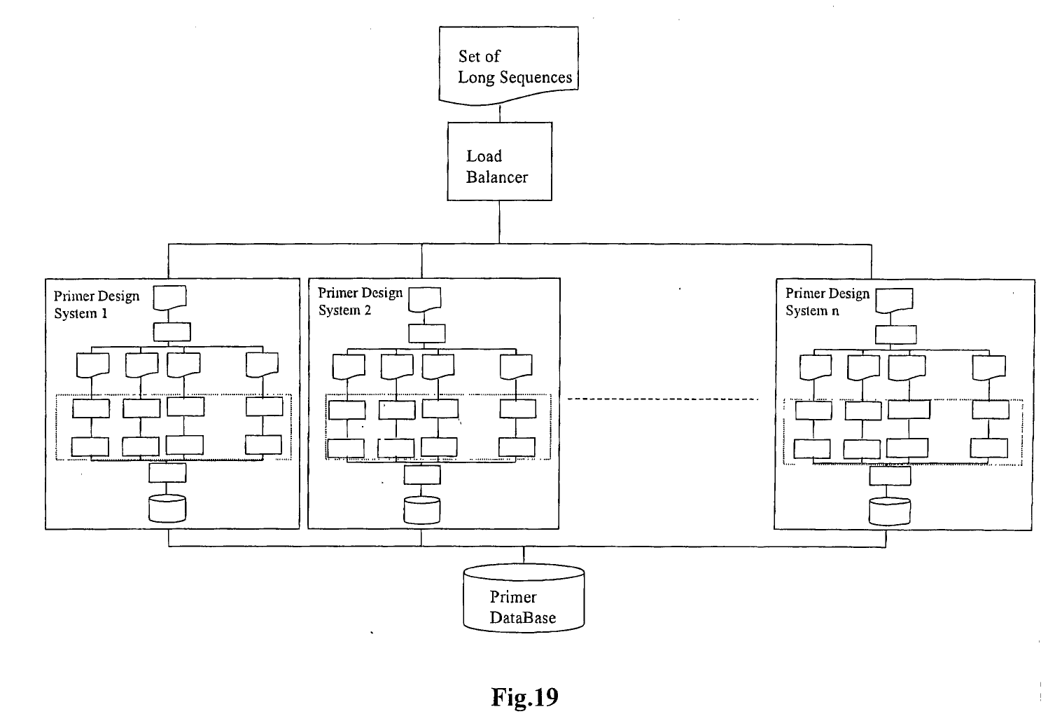

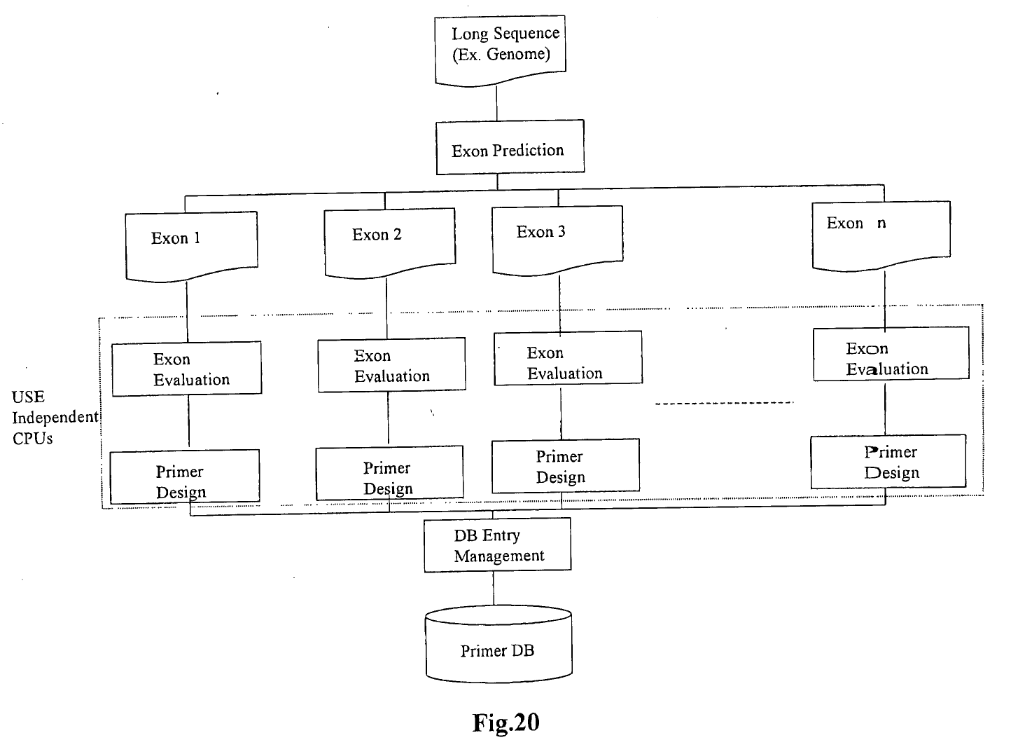

This invention designs plural primers based upon not only plural exons (FIG. 20) but also plural genomic DNA sequences (FIG. 19). Since the appropriate parameters (such as CG contents, etc) are fixed empirically for designing usable primers as many as possible. Sometimes, deficient primers are designed or the program cannot find suitable regions for primer design. However, the invention realizes extremely higher-throughput comparing to the conventional protocol by fixing the parameters empirically. The system shown in FIG. 19 allows 95 out of 96 primers produced single bands in the experiment. This result shows the primer design system of this invention is applicable to improve the practical primer design for PCR experiments.

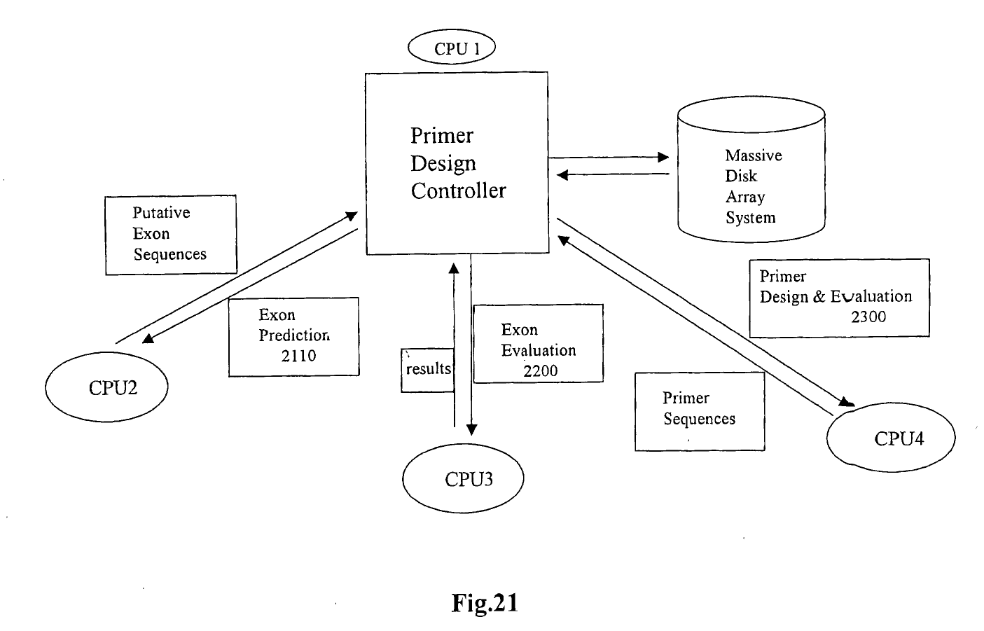

This system can design plural primer pairs concurrently by using of multiple CPUs. FIG. 20 shows the process flow of the primer design. The primer design process in this system consists of the functions of exon prediction, exon evaluation and primer design. Each function can be processed by independent CPUs because the functions require neither any parameters modified by nor the communications with the other functions (FIG. 21). If each process needs to use specific parameters and to store the intermediates, they can keep them in separate files or directories of common storage (disk arrays). If the web is served by one of the CPUs, the user can construct the control system through intranet by using of the common interfaces including cgi. By integrating the results of the calculations of each function, this system can construct the primer database with the huge number of primer sequences in a practical time-frame in contrast to the conventional interactive-based primer design system.

Based on the same characteristics, a plurality of the primer design systems shown in FIG. 19 are arranged t work in parallel in FIG. 19. Large-scale system combined multiple systems operates more efficiently to provide high throughput.

When the conventional primer design system is employed, the conditions of the primer design are inspected and checked one by one by a researcher. As a result, it takes at least 5 minutes even for a skilled researcher to design the right primer pairs. In the system of the invention, several sets of parameters for primer design software (Primer 0.5 or Primer 3), such as primer lengths, numbers of GC in the 3′ end of primers, target Tm, non-specific binding regions including loop structures, are arranged based on experiments including actual PCR amplification with resultant primer pairs. Primers designed for a kind of bacterium of intestinal flora was examined in the inventors' laboratory. For example, 96 primer pairs were designed from the genomic sequence of the bacterium and performed with PCR amplification. As a result, 95 positive amplifications were detected and all were single bands. The remaining one sample did not show any band, and it is not clear if the cause of the failure of the amplification is solely an error of the primer design by this system.

In addition, because this primer designs plural primer pairs from the given long sequences including genomic sequences, partial sequences and primers can be checked against the DNA sequences. If the extracted sequences (ex. exons) appear several times in the given sequences, the extracted sequences contain the repeated sequences such that such a region is not appropriate for primer design. This is the advantage absent form the conventional primer design system based on the interactive operation.

Rather than one DNA sequence, a plurality of DNA sequences are processed in parallel as shown in FIG. 19 with plural primer designing systems 1 through n. A load balancer is added to balance the workload of the plural primer designing systems. As shown in FIG. 20, each primer design system processes a plurality of fragments 1 through n in parallel with plural CPUs working simultaneously so as to predict plural exons from each fragment. The putative exons are processed in bulk for each steps of FIG. 9. Also see FIG. 21, the putative exons are processed for exon prediction 2210 by CPU2, then the data are processed for fragment evaluation 2220 by CPU2, then the data are processed for primer design 2230 by CPU4 so as to output primers to be saved in a primer database. In other words, each processor executes different instructions. The processors are communicate with the CPU1, which distributes work to the others and collects results from them, in order to be able to cooperate in carrying out the whole processing.

In another embodiment of the invention, each of the putative exons are processed via the exon prediction 2100, exon evaluation 2110, and primer design 2200 consecutively with time overlaps between any two of the putative exons. In other words, all processors execute the same sets of instructions simultaneously but each at a different stage/step. In this case, the CPUs run completely independently.

As mentioned, the present invention conducts at least two layers of sequence specificity checks as shown in FIG. 22. First of all, the predicted exons are evaluated according to at least one of the following conditions (a)-(d).

(a) predicting with an exon predicting program;

(b) from an EST database;

(c) SNPs derived from an EST database and having a genotyping potential;

(d) from at least one protein database, such as cDNAs obtained from clustering and alignment (CAT) analysis and having no known function.

Secondly, the designed primers are selected with at least one of the following conditions (a)-(e) in Steps 1710-1725, then evaluated for specificity with BLAST in Steps 1730-1775 according to FIG. 11.

(a) meeting a predetermined base length: 20-28 bps;

(b) GC content: 50-60%;

(c) Tm=50-80° C. and |ΔTm|<20° C.;

(d) located as close to the 5′ end or the 3′ end as possible;

(e) including non-specific binding regions, such as loop structures.

For each full sequence (a primer plus the target region), BLAST searches are conducted via a repeat database in the step 1730 and a genome database in the step 1735. If the full sequences for either the forward primer and the reverse primer specific enough, the primer pair is selected in the step 1745. If either of the full sequences are suspected to contain any undesired sequences, such as E. coli contamination in the step 1750, a BLAST search is conducted to see whether the suspicious sequence is undesirable so as to screen out the primer pair. Thereafter, for each primer, BLAST searches are conducted via a repeat database in the step 1760 and a genome database in the step 1765 to ensure it is specific enough. Then a specificity check, i.e., justification checks on each multiplication region in the same DNA which contains the exact sequence as the primer but positioned elsewhere, is conducted for each primer pair in the step 1775 to ensure the primer structure doesn't appear too often in the same DNA sequence.

The actual performance of the system is tested with three DNA sequences of S. cerevisiae and C. elegans on an operation system IRIX 6.5 with one CPU of 400 MHz and memory of 512 MB. C. elegans is about as primitive an organism that exists which nonetheless shares many of the essential biological characteristics that are central problems of human biology. Saccharomyces cerevisiae, better known as baker's yeast, is the first unicellular eukaryotic organism to be completely sequenced. For example, the starting time of the program of the invention to process YFL039C was 18:16:52 when the processing cut out a FASTA file from the results of the primer design, and the ending time was 18:17:08 when the system finishes sequence specificity check. In case of C. elegans, BLAST search is performed to E. coli as well. C. elegans is fed by E. coli because sequences obtained from E. coli are often contaminated at the PCR amplification. Based upon the table 1, the elapsed time (processing time) for C. elegans is longer than that for of S. cerevisiae, and the BLAST data size for C. elegans is larger than that for of S. cerevisiae since the genome size of C. elegans is larger.

TABLE 1

| | | length | Primer | | | | start | end | elapsed |
|---|---|---|---|---|---|---|---|---|---|
| | DNA seq. | bps | Num | ◯ | X | | time | time | time |
| S. cerevisiae | YFL039C | 1,436 | 5 | 3 | 0 | 3 | 18:16:52 | 18:17:08 | 00:00:16 |
| | YMR250W | 1,758 | 5 | 0 | 5 | 5 | 18:20:41 | 18:21:06 | 00:00:25 |
| | YML059C | 5,040 | 5 | 0 | 5 | 5 | 18:23:14 | 18:23:35 | 00:00:21 |
| C. elegans | C44C11 | 639 | 5 | 2 | 0 | 2 | 18:27:39 | 18:32:37 | 00:04:58 |
| | C54D2 | 912 | 5 | 2 | 0 | 2 | 18:50:44 | 18:55:14 | 00:04:30 |
| | F17C8 | 4,566 | 5 | 2 | 3 | 5 | 19:16:21 | 19:31:36 | 00:15:15 |

The number of plate wells and the number and type of primers contained in the plates are not particularly limited. Plates may have wells which do not contain solutions with primers, or all the wells may contains solutions containing primers. Each well may contain solutions with one type of primer, or solutions with 2 or more types of primers. Although different wells usually contains solutions with different types of primers, different wells may also contain solutions with the same types of primers.

For comprehensive DNA analysis, 75 or more types of solutions in all should be contained per plate. For even higher analyzing efficiency, 80% or more of the total number of wells should contain different solutions.

Commercially available 96-well plates, 384-well plates, and the like can be used as the plates with a plurality of wells. In such cases, PCR can be carried out for large amounts of sample DNAs with each plate having 76 or 307 kinds of solutions with primers.

The composition of the solutions containing the primers is not particularly limited, provided that PCR can be carried out in the solutions. Since the PCR reaction solution usually contains H₂O, PCR buffer, MgCl₂, dNTP mix, Taq polymerase, and the like in addition to primers and template DNA, the solutions containing the primers may contain 1 or more the above-mentioned contents thereof.

The primer concentration in the solution can be selected as desired, but is preferably between 10 and 100 pmol/μL. Conventionally, the concentration is a thick one at the order of micromol/mL, and is diluted for use, but when the concentration is about 10 to 100 pmol/pL from the beginning, the user can use it directly without dilution. The solution should also contain no enzymes that degrade the primers (such as DNase).

The plates may also comprise lids, films or the like to cover the wells so as to prevent the primer solutions in the wells from becoming mixed with each other during distribution. When the film is one that can be broken by a robot liquid handling capillary, an advantage is that it can be mounted on the robot as is.

### EXAMPLE 1

A relatively new sequence which had not been analyzed very much was selected from the sequence database of Chromosome 21 publicly disclosed on the WWW (ERI Chromosome 21 Sequence Database: http://www-eri.uchsc.edu/chr21/c21index.html). Processing this sequence by an existing exon predicting programs (program A and B) resulted in the prediction of four sequences (exon 1: SEQ ID NO: 1; exon 2: SEQ ID NO: 2; exon 3: SEQ ID NO: 3; exon 4: SEQ ID NO: 4) as exon nucleotide sequences. The machine used to predict the exons was a SUN Ultra 60 ( 2 GB memory), and the prediction time was about 5 minutes per sequence with program A (access via NCBI BLAST mail server) and about 10 minutes with program B (run BLAST on a local server).

Partial sequences meeting the following extraction conditions were extracted from each of the predicted exon nucleotide sequences as the primer pair:

(1) base length: 20 to 28 bps;

(2) GC content: 50 to 60%;

(3) Tm: 50 to 80° C.; |ΔTm|: below 20° C.; and

(4) located as close as possible to the 5′ end or 3′ end.

A Blast search was performed on the GenBank database with the extracted partial sequences as the query, and an Identities value of 50% or lower was selected to screen for partial sequences of high specificity. When screening of partial sequences of even higher specificity is desired, the Identities value can be set lower (such as 30% or lower), and when other conditions are to be prioritized at the expense of a certain degree of specificity, a higher identities value (such as 70% or more) can be set.

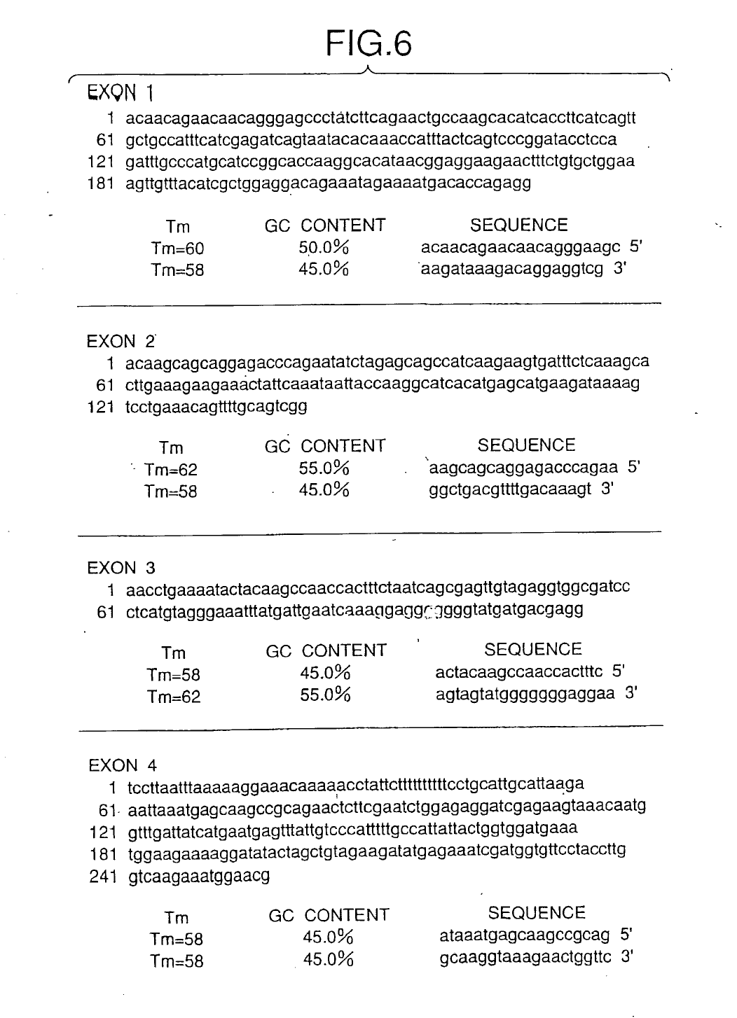

As a result, the partial sequences given in SEQ ID NOS: 5 and 6 were extracted from exon 1 (SEQ ID NO: 1), the partial sequences given in SEQ ID NOS: 7 and 8 were extracted from exon 2 (SEQ ID NO: 2), the partial sequences given in SEQ ID NOS: 9 and 10 were extracted from exon 3 (SEQ ID NO: 3), and the partial sequences given in SEQ ID NOS: 11 and 12 were extracted from exon 4 (SEQ ID NO: 4) (FIG. 6).

### EXAMPLE 2

The time needed to execute the following patterns I through III one thousand times was calculated. A SUN Ultra 60 (2 GB memory) computer capable of locally running the necessary programs was used for each of the patterns.

Pattern I

Only primer designing was carried out. Pattern I involved running a process for extracting partial sequences from the predetermined template DNA sequence A1 based on primer design software corresponding to the partial sequence extraction processor 403. The partial sequence extraction conditions were as follows.

(1) base length: 20 to 28 bps;

(2) GC content: 50 to 60%;

(3) Tm: 50 to 80° C.; |ΔTm|: below 20° C.; and

(4) located as close as possible to the 5′ end or 3′ end.

Pattern II

For pattern II, exons were evaluated, and primers were then designed. For pattern II, exons were screened based on selected conditions from previously prepared exon database 307, template DNA sequence A1 was transferred through the input 401 to the exon prediction processor 403, and the process for predicting exons was run based on primer design software corresponding to the exon prediction processor 403. The exon evaluation conditions are given below. The partial sequence extraction conditions were the same as for pattern I.

(1) exon length: 300 bps or less

(2) exons predicted by an exon predicting program

(3) found in EST database, and expression confirmed

(4) unknown function (not found in protein database)

(5) SNP potential (variation in EST database)

Pattern III

After the exon prediction, exons were screened, and primers were then designed. For pattern III, exons were predicted using software corresponding to the exon predicting program 304 from genomic DNA sequences 303, the output exon sequences 305 were compiled into a database 307 through a sequence input interface 306, exons were screened in the exon database 307 on the basis of the set conditions, the DNA sequence A1 was transferred through the input 401 to the exon prediction processor 403, and the process for extracting primers was run by primer design software . The exon evaluation conditions were the same as for pattern II. The partial sequence extraction conditions were the same as for pattern I.

Table 2 shows the results of calculations for the time needed to run patterns I through III one thousand times, respectively. In Table 2, “T1” represents the time (minutes) needed for exon prediction, “T2” represents the time (minutes) needed for exon evaluation, and “T3” represents the time (minutes) needed for primer design.

TABLE 2

| | I | II | III |
|---|---|---|---|
| T1 (min) | 0 | 0 | 1244.8 |
| T2 (min) | 0 | 598.2 | 598.2 |
| T3 (min) | 49.8 | 49.8 | 49.8 |
| Calculation time (min) | 49.8 | 648 (10.8 H) | 1892.8 (31.55 H) |
| needed to design 1000 | | | |
| primers | | | |
| Calculation time when | 1.0 | 13.0 | 37.9 |
| simultaneously treated by | | | |
| parallel processes (ex. 50) | | | |

The results of Table 2 show that the primer design system of the present invention can be used to design about 5000 primer pairs per day through parallel computers, which means about 150,000 primers could be sufficiently prepared in a year.

The primer design system of the present invention allows a plurality of primers capable of hybridizing exons to be efficiently prepared. The plurality of primers capable of hybridizing specifically to exons can be used in DNA analysis, allowing large amounts of sample DNAs to be efficiently analyzed all at once. It is particularly useful for high throughput screening. During DNA analysis, a computer-readable storage medium in which are recorded data for the plurality of primers capable of hybridizing specifically to mutually different DNAs and genetic data for DNA fragments amplified by PCR using these primers can be used to make such DNA analysis easier.

The principles, preferred embodiments and modes of operation of the present invention have been described in the foregoing specification. However, the invention which is intended to be protected is not limited to the particular embodiments disclosed. The embodiments described herein are illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A primer design system, comprising:
means for selecting at least one genomic DNA nucleotide sequence from a database including a plurality of DNA nucleotide sequences;
means for predicting a plurality of exons of said selected DNA nucleotide and for storing positions of the predicted exons;
means for simultaneously designing a plurality of primer pairs by using each of the predicted exons as a template; and
means for automatically collating said plurality of primer pairs with said predicted exons and the DNA nucleotide sequence.

2. A primer design system according to claim 1, further comprising means for selecting a plurality of primer pairs meeting certain selection conditions from the designed primer pairs.

3. A primer design system according to claim 2, said selection conditions include at least one of a predetermined base length, a range of GC content and a range of Tm.

4. A primer design system according to claim 1, further comprising means for evaluating specificity of each designed primer or primer pair.

5. A method for designing primers, comprising the steps of:
selecting at least one DNA nucleotide sequence from a genomic DNA database;
predicting a plurality of exons of said selected DNA nucleotide;
simultaneously designing a plurality of primer pairs by using each of the predicted exons as a template; and
automatically collating said plurality of primer pairs with said predicted exons and the DNA nucleotide sequence.

6. A method for designing primers according to claim 5, further comprising a step of selecting a plurality of primer pairs meeting certain selection conditions from said plurality of designed primer pairs, wherein said extraction conditions include at least one of a predetermined base length, a GC content, Tm.

7. A method for designing primers according to claim 5, further comprising a step of evaluating specificity of each designed primer or primer pair.

8. A primer design system according to claim 1, further comprising randomly dividing fragments of a genomic DNA as templates for exon prediction.

9. A primer design system, comprising:
means for selecting at least one genomic DNA nucleotide sequence from a database including a plurality of DNA nucleotide sequences;
means for predicting a plurality of exons of said selected DNA nucleotide and for storing positions of the predicted exons;
means for designing a plurality of primer pairs by using each of the predicted exons as a template; and
means for evaluating specificity of each designed primer or each designed primer pair.

10. A primer design system according to claim 9, wherein the means for evaluating specificity evaluates each designed primer by conducting BLAST searches for a full sequence of the primer via at least one repeat database and at least one genome database.

11. A primer design system according to claim 9, wherein the means for evaluating specificity evaluates each designed primer by conducting a BLAST search for any undesirable sequence contained therein.

12. A primer design system according to claim 9, wherein the means for evaluating specificity evaluates each designed primer pair by conducting justification checks on each multiplication region in the DNA which contains an exact sequence as the primer but positioned elsewhere on the DNA.


SEQUENCE LISTING (12 sequences)

SEQ ID NO: 1 - DNA, 227 bp, Homo sapiens
acaacagaacaacagggagccctatcttcagaactgccaagcacatcaccttcatcagttgctgccatttcatcgagatcagtaatacacaaaccatttactcagtcccggatacctccagatttgcccatgcatccggcaccaaggcacataacggaggaagaactttctgtgctggaaagttgtttacatcgctggaggacagaaatagaaaatgacaccagagg

SEQ ID NO: 2 - DNA, 143 bp, Homo sapiens
acaagcagcaggagacccagaatatctagagcagccatcaagaagtgatttctcaaagcacttgaaagaagaaactattcaaataattaccaaggcatcacatgagcatgaagataaaagtcctgaaacagttttgcagtcgg

SEQ ID NO: 3 - DNA, 114 bp, Homo sapiens
aacctgaaaatactacaagccaaccactttctaatcagcgagttgtagaggtggcgatccctcatgtagggaaatttatgattgaatcaaaggagggggggtatgatgacgagg

SEQ ID NO: 4 - DNA, 256 bp, Homo sapiens
tccttaatttaaaaaggaaacaaaaacctattcttttttttttcctgcattgcattaagaaattaaatgagcaagccgcagaactcttcgaatctggagaggatcgagaagtaaacaatggtttgattatcatgaatgagtttattgtcccatttttgccattattactggtggatgaaatggaagaaaaggatatactagctgtagaagatatgagaaatcgatggtgttcctaccttggtcaagaaatggaacg

SEQ ID NO: 5 - DNA, 20 bp, Homo sapiens
acaacagaacaacagggagc

SEQ ID NO: 6 - DNA, 20 bp, Homo sapiens
aagataaagacaggaggtcg

SEQ ID NO: 7 - DNA, 20 bp, Homo sapiens
aagcagcaggagacccagaa

SEQ ID NO: 8 - DNA, 20 bp, Homo sapiens
ggctgacgttttgacaaagt

SEQ ID NO: 9 - DNA, 20 bp, Homo sapiens
actacaagccaaccactttc

SEQ ID NO: 10 - DNA, 20 bp, Homo sapiens
agtagtatgggggggaggaa

SEQ ID NO: 11 - DNA, 20 bp, Homo sapiens
attaaatgagcaagccgcag

SEQ ID NO: 12 - DNA, 20 bp, Homo sapiens
gcaaggtaaagaactggttc